(19) United States
(12) Patent Application Publication
(10) Pub. No.: US 2008/0015484 A1
(43) Pub. Date: Jan. 17, 2008

(54) Apparatus and method for cleaning and disinfecting body piercings

(76) Inventor: Joan A. Wolfensberger, Chicago, IL (US)

Correspondence Address: BRINKS HOFER GILSON & LIONE, P.O. BOX 10395, CHICAGO, IL, 60610, US

(21) Appl. No.: 11/487,048

(22) Filed: Jul. 14, 2006

(51) Int. Cl. A61M 35/00; A61F 13/00

(52) U.S. Cl. 604/2; 602/53

(57) ABSTRACT

A device for applying a treatment liquid to a wound caused by piercing of a body part is described. The device includes one or more receptacles having a well therein, suitable for accommodating a foam tablet. The receptacles are joined by a strap so that the well openings face in the substantially the same direction when the device is in an open position. An open-cell foam tablet is inserted into the well so that a portion of the foam tablet extends above a top end of the well. The strap may be a hinge or flexure so that the device may be bent about a central region of the strap. A treatment liquid for cleaning or disinfecting the wound may be charged into the foam, and the wound treated by bending the device position so that the extended foam portions are brought into contact with the area to be treated.

### TECHNICAL FIELD

The present application relates to an apparatus and method for treating a pierced human body part with a cleaning or disinfecting liquid.

### BACKGROUND

The earlobe is the most common body piercing. Other common places pierced include the ear cartilage (the hard part around the upper edge of the ear), eyebrow, nose, tongue, lip, belly button, nipples and genitals. Piercing is not regulated in most states, and may be done in tattoo parlors or in kiosks in shopping malls. For ear piercing, a type of gun is used having sterilized insert parts, or a sterilized needle may be used, particularly in the case of other body parts. In each instance, it is common to insert a temporary stud or ring in the wound hole to keep the piercing open during the healing process. As piercing is not considered a medical procedure, the after piercing care advice tends to depend on the source of the advice.

Body piercings take time to heal, even with diligent care. Depending on the site of the piercing, expected times for normal healing may vary and special care is required. For example, a conventional ear piercing may take 6-8 weeks to heal, and a piercing through the upper rim (helix) of the ear goes through cartilage and takes an average of 2-3 months to heal. It must be cleaned 2-3 times daily during that time. Tongue piercings should be cleansed at least a dozen times per day with antiseptic mouthwash for 6-8 weeks. Navel piercings should be cleaned twice daily for at least 9 months and daily in the shower thereafter.

For ear piercings, recommended cleaning solutions include mild liquids, such as 0.9% sterile isotonic solution of “normal saline” or prepared saline; mild liquid anti-microbial or germicidal soap such as Provon or Satin or other fragrance free mild anti-microbial soap; some proprietary cleaning liquids may include benzalkonium chloride. Similarly, there are a number of other solutions used for topical medicinal purposes that are commonly recommended against, such as alcohol, hydrogen peroxide, Betadine, Hibiclens or ointment. The selected cleaning solution is applied to the pierced area two or more times a day using a cotton ball or bud.

The saturated cotton ball or bud tends to express liquid when pressed by the fingers, or pressed against the ear lobe or other body part. Commonly this results in the liquid dripping on the user, running down the hand onto the arm, or dripping on clothing.

### SUMMARY

An apparatus and method for cleaning or disinfecting body piercings is disclosed. When in a closed configuration, the apparatus has the form of a clam or receptacle, with the two “shells” of the clam joined by a flexure. Each of the shells has a well structure formed therein, the wells opposing each other when the apparatus is in a closed position. Each of the wells is sized and dimensioned to accept a tablet having a circular, oval, rectangular or similar shape and being fabricated from a foam or foam-like material. When inserted in the well, the foam tablet projects above a plane defined by a surface of the shell facing the opposing shell so that foam tablet in each well may contact a body part such as an ear lobe when the clam shell is in a closed state with the ear positioned between the shells after the tablets have been inserted. The foam material is selected as at least a partially-open-cell foam so as to be permeable to the cleansing solution, and of a material compatible with the cleansing solution. The periphery of the foam tablet is sized so that it forms an interference fit with a side wall of the well, holding the foam tablet in place when in use, but permitting a low-force extraction of the foam tablet after use.

In an aspect, a kit may be provided, including, in addition to the cleaning apparatus, one or more of foam tablets, cleaning solution, cleaning solution dispenser, and packaging

In another aspect, the exterior surface of the apparatus may have ridged or raised stripes or a roughened area formed so as to facilitate gripping the apparatus even when wet.

In yet another aspect, the apparatus may be sized and dimensioned so as to be compatible with the use thereof to cleanse or disinfect piercings of other body parts, including, but not limited to, the nose and ear cartilage.

### BRIEF DESCRIPTION OF THE DRAWINGS

FIG. 1 is an elevation (A) and a cross-sectional (B) view of a cleaning device;

FIG. 2 A-C show alternative cross sectional views of a receptacle and D-E show alternative top views of a receptacle; and

FIG. 3 shows the cleaning device applied to an earlobe.

### DETAILED DESCRIPTION

Exemplary embodiments may be better understood with reference to the drawings, but these embodiments are not intended to be of a limiting nature. Like numbered elements in the same or different drawings perform equivalent functions.

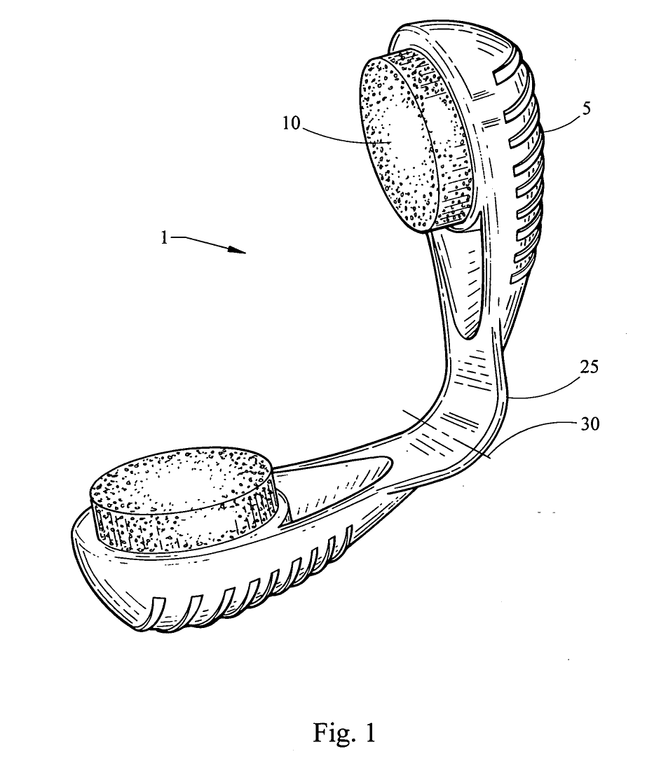

An apparatus and method for applying a cleaning or disinfecting liquid to a wound associated with piercing a body part is described. In an example, sized and dimensioned for use in cleaning pierced ears, an apparatus such as that shown in FIG. 1 may be used. The cleaning device I may be formed of molded plastic, metal or other material. When laid flat a maximum dimension is approximately 5″ in length, and a maximum width is 1¼″ in. Each end of the cleaning device 1, which may be termed a clam shell or receptacle 5, has a well 15 or recess sized and dimensioned to accept a foam tablet 10. The two clam shells are joined with a strap 25 about ¾″ in width, which may be made of the same material as the clam shells, and be sufficiently flexible to act as a flexure hinge. Alternatively, a hinge may be provided at the mid-point 30 of the strap.

The clam shell portion 5 may be made of a moldable material such as LHTA Injection Molding Homopolymer polypropylene resin PP3429 available from Total Petrochemicals USA, Inc. (Houston, Tex.), although many suitable moldable materials exist and may be used as alternatives. The strap 25 may be made of the same or similar materials as the receptacle 5, and may be molded integrally with the receptacle 5. Any material that is flexible and durable enough to flex repeatedly at the mid point 30 of the strap 25 without breaking may be used. The rigidity required is primarily determined by the perceived handling properties when used. Materials such as polypropylene (PP), polyethelyne (PE), styrene acrylonitrile (SAN), polyethelyne terephalate (PET), or soft polyvinyl chloride (PVC), for example, may be considered for use. The material may transparent, translucent or opaque, and may be colored for esthetic reasons.

The foam (or foam-like) tablet 10 (hereinafter “foam tablet”) may be fabricated from material with good liquid wicking properties (such as an open-cell foam) which may die-cut from a sheet, be soft to the touch, and flexible enough to conform around the shape of a temporary or starter earring without a tendency to snag. In an example, the foam tablet 10 is made of Ultra Fine available from E. N. Murray Co (Denver, Colo.). The material is a high density; fine pore, fully open-cell polyester polyurethane formulated with a proprietary additive that gives Ultra Fine a velvet-like, luxurious feel. Similar soft foam materials with good wicking characteristics and that are inert to mild antiseptic agents may also be used. Wicking is the phenomenon of absorption of a liquid into a material by capillary action. The foam tablet 10 may have an impervious film or coating on the face opposite the free face 40 so as to minimize the leakage of the liquid which may be charged into the foam tablet 10 for the treatment process.

The foam tablet 10 has a peripheral shape and size such that a slight interference fit may be achieved with the side surface 20 of the well 15 in the clam shell 5, and a height may be such that a face 40 of the foam tablet 10 may project from the surface 35 of the receptacle 5 so as to envelop the opposing portion of a starter earring inserted in the piercing without the clam shell 5 substantially contacting the ear part.

The cleaning device 1 may be used with a variety of liquids that have been, or may be, recommended for topical application. For the treatment of ear piercing wounds, for example, the following have been recommended for use: 0.9% sterile isotonic solution of “normal saline” or prepared saline; mild liquid anti-microbial or germicidal soap such as Provon or Satin or other fragrance free mild anti-microbial soap; some proprietary cleaning liquids may include benzalkonium chloride (1:1000), available, for example as EAR care (Piercing Pagoda, Inc., Lehigh Valley, Pa.). The list is not inclusive and other products may be suitable, or be recommended in the future. The liquid may be selected and provided by the user, or may be provided as part of a kit including one or more of a container holding the liquid, a dispenser for the liquid, which may be a part of the container or be separate therefrom, a supply of foam tablets, or the cleaning device.

In an aspect, the foam tablet 10 may be charged with the liquid by use of a pipette, commonly also called a medicine dropper, or by squeezing a soft-sided container having a nozzle, pouring or otherwise dispensing the cleansing solution. Certain of the dispensing techniques are capable of dispensing a calibrated or controlled amount of the liquid and may lead to more effective use of the cleaning device, however the amount of liquid dispensed may be effectively judged by the user after a few trials. The use of a foam tablet and a dispenser reduces the user chance of coming in contact with the cleansing solution during use and may optimally saturate the foam tablet to cleanse the puncture site, while minimizing dripping. Alternatively, a foam tablet pre-packaged and pre-saturated with the liquid and having a removable protective film on an outward facing side may be provided. In another alternative, the pre-saturated foam tablets are packaged in bulk in a container, or packaged in pairs in a sealed plastic bag.

The cleaning device 1 is used by placing or replacing the foam tablet 10 in the clam shell well 15 with a unused foam tablet 10, and if the foam tablet 10 is not pre-saturated with liquid, charging the foam tablet 10 with an appropriate volume of the liquid. The cleaning device 1 is folded about the flexure portion 30 so as to envelop the pierced ear site and the earring. The user applies pressure to clam shells 5 either directly or indirectly, by pressing against the opposing portions of the folded strap 25. By adjusting the pressure applied by the fingers to the exterior of the cleaning device 1, the user can cause the foam tablets 10 to press against the ear lobe and the earring so as to express or discharge some of the liquid held by the foam tablet 10 onto the ear lobe and ear ring, thus applying the liquid to the area to be treated.

The user may apply the cleaning device to the area to be treated in this manner for approximately 1 minute. The time duration of application is not critical, and is mentioned as a typical cleansing time recommended by various organizations, whose recommendations may be viewed by searching on the World Wide Web (WWW). Once the ears are cleansed, the foam tablets 10 can be discarded. Desirably, the user will replace each used foam tablet 10 with an unused foam tablet 10 and perform the procedure on the other ear.

Similarly, the cleaning device may be used to treat piercing wounds of the nose, the lips, etc. The shape and size of the cleaning device may be varied to suit the sizes of the objects to be treated and to minimize interaction with other adjacent body parts. Where access to both sides of the body part is not convenient, such as with naval piercing, one of the claim shells may be held against the ring or bar inserted in the naval piercing would so as to dispense a controllable amount of the treatment liquid.

In an example, a cleaning device 1, shown in FIG. 1 includes one or more receptacles 5 for receiving a foam tablet 10 and joined by a strap 25. The strap 25 may be configured such that it may be bent or flexed about a mid-point 30 between two receptacles 5. The strap may be substantially flat when in the unflexed or open position, or bent at an angle about the mid-point 30 as shown in FIG. 1, suggesting to the user the direction in which the bending should continue during the use thereof. A foam tablet 10 is shown inserted in a well 15. This may be seen in FIG. 1B where the foam tablet 10 is shown displaced from the well 15. The foam tablet 10 forms an interference fit with the periphery 20 of the well 15 and the height of the foam tablet 10 is such that a portion of the foam tablet protrudes from the well 15 above a surface 35 of the receptacle 5; the outward facing portion of the foam tablet 10 has a free face 40. The free faces 40 of the foam tablets 10 face in substantially the same direction when the cleaning device 1 is in the open position of FIG. 1. That is, the axes of the wells 15 point in substantially the same direction, deviating from being in the same direction primarily due to the bending of the strap 25 about the mid-point 30. In an alternative, the flexible aspect of the strap 25 may be replaced, in whole or in part, by a hinge disposed at the mid-point 30, so as to permit the angle formed by the strap on either side to be made more of an acute angle during the use of the cleaning device 1.

To improve the grip of a person on the cleaning device 1, ridges 50, grooves or roughened areas may be provided on the exterior surfaces of the cleaning device 1. This may permit the user to better grip the cleaning device with the fingers of the hand during use. Such ridges, grooves of roughness may be provide also on the interior peripheral surface 20 of the well 15 to improve the retention of the foam tablet 10.

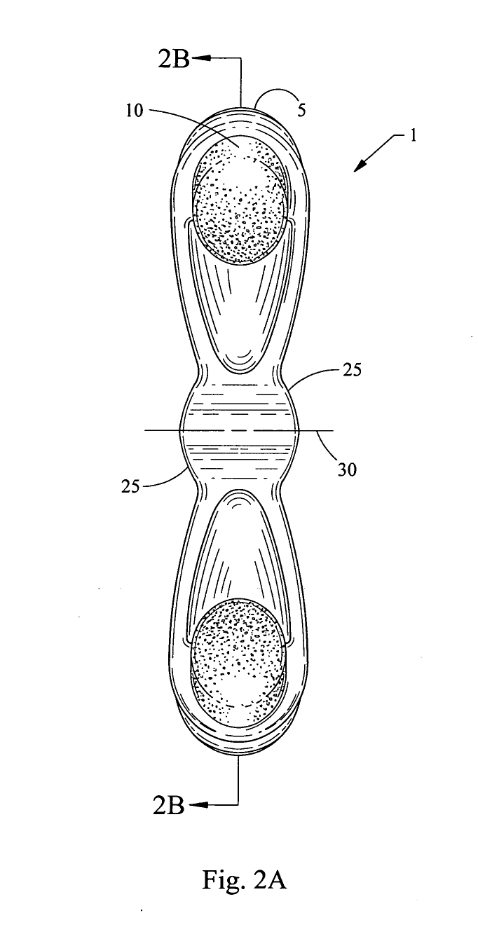

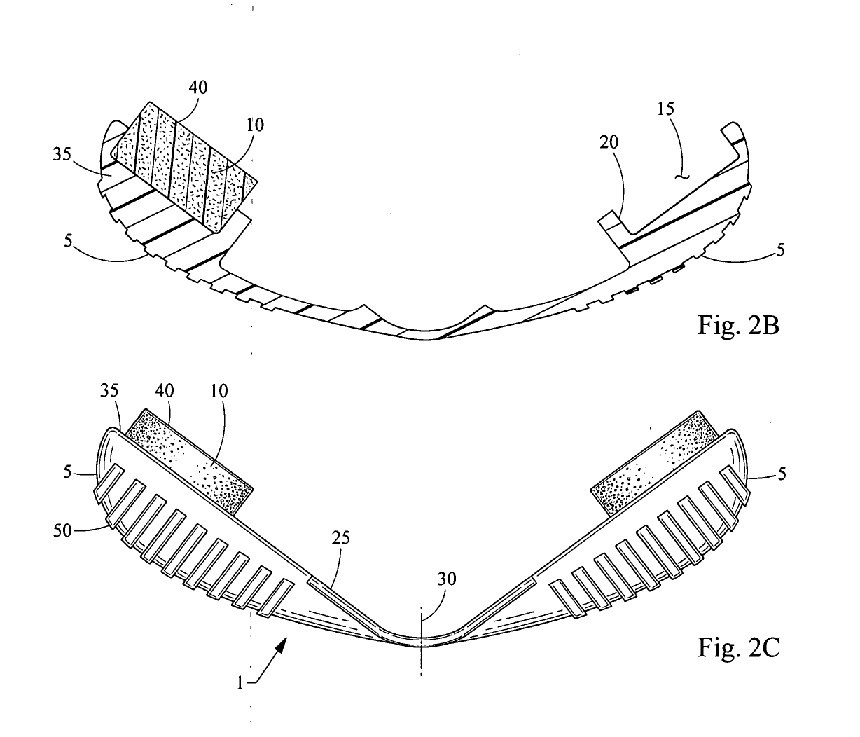

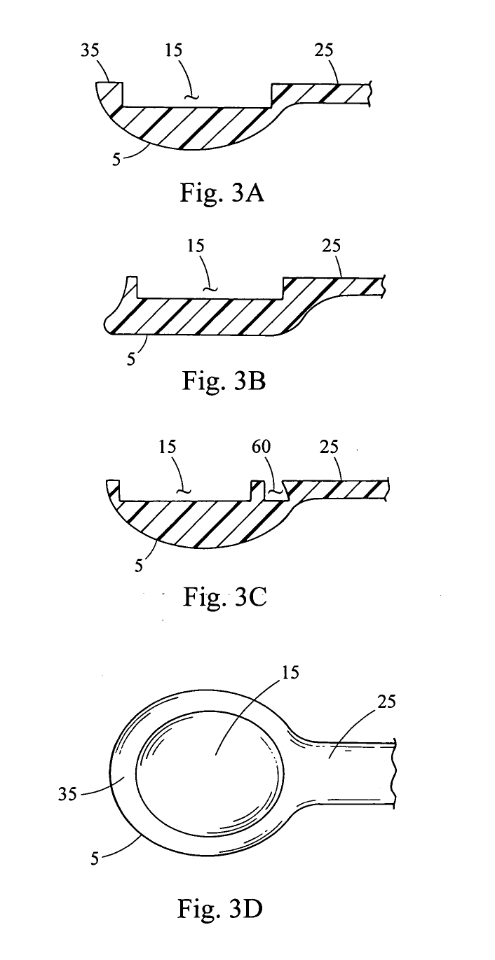

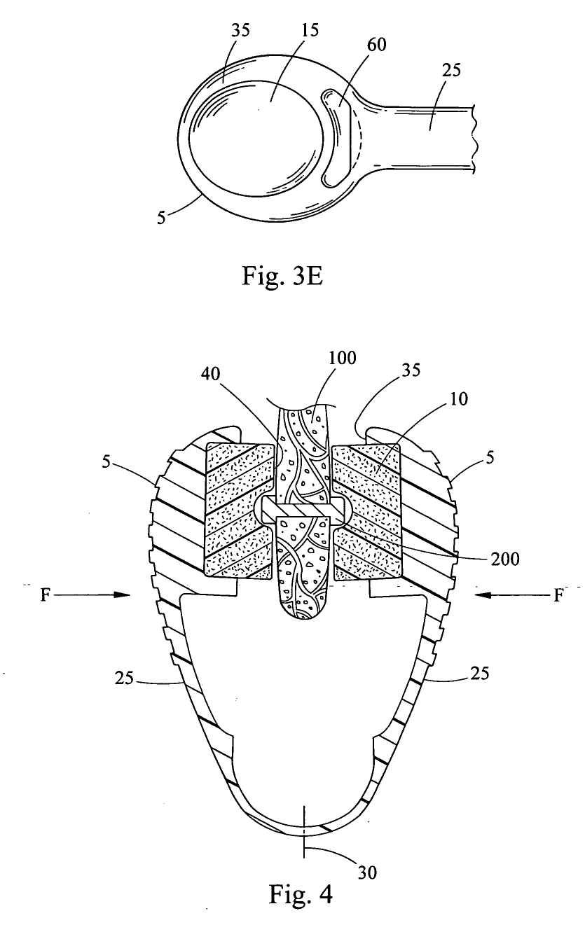

The shape of the receptacle 5, the strap 25, and the well 15 may be adapted to suit ergonomic considerations, depending on the body part to be treated, so as to facilitate bringing the free surface 40 of the foam tablet 10 in contact with the area to be treated. FIG. 2 shows a number of alternative shapes of the receptacle 5 and the well 15, where the shape of FIG. 2A corresponds to that of the example of FIG. 1, as shown in cross section. The example of FIG. 2B shows a configuration where the extension of the receptacle 5 in the direction opposite to that of the strap 25 is minimized, so as to avoid interference with the attachment region of the ear lobe to the body. In FIG. 2C, a trough 60 is formed in the surface 35 of the receptacle 5. When the cleaning device 1 is applied to an ear lobe 100 as shown in FIG. 3, the face 35 of the receptacle is approximately vertical, and any liquid which is in excess of that necessary for treatment may run downwards due to the force of gravity and collect in the trough 60. This catchment trough 60 may minimize any dripping from the cleaning device 1, even if the user is somewhat careless in the control of application pressure.

FIG. 2D is a top view of a receptacle 5 showing a well 15 having an elliptical shape. Other cross sectional shapes, in addition to circular, may be ovoidal, triangular, rectangular and the like. FIG. 2E shows an example of the receptacle 5 having a catchment trough 60. Such a structure may be combined with any of the shapes of receptacle and well. Although not shown, ridges, grooves or surface roughness may be applied to the lower surface of the strap 25.

FIG. 3 shows the cleaning device 1 applied to an ear lobe 100 having a bar 200 inserted in a piercing. The cleaning device 1 is bent or folded about the mid-point 30 so that the free faces 40 of each of the foam tablets 10 comes in contact with a surface of the ear lobe 100 in a region where the bar 200 has been inserted. Applying pressure in the direction of the arrows F compresses the foam tablets 10 against the earlobe 100 and causes the expression of the liquid treatment material which has previously been charged into the foam tablets 10. Although the force F is shown as being applied directly to the receptacles 5, it may be applied indirectly by applying force to the opposing portions of the strap 25. The cleaning device 1 maintained in the closed configuration shown in FIG. 3 for the duration of the treatment period, which may typically be about one minute for each ear.

It is therefore intended that the foregoing detailed description be regarded as illustrative rather than limiting, and that it be understood that it is the following claims, including all equivalents, that are intended to define the spirit and scope of this invention.

What is claimed is:

1. An apparatus for applying a liquid to a body part, comprising:
a pair of receptacles sized and dimensioned to accept a foam tablet in a well thereof;
a strap joining the pair of receptacles

2. The apparatus of claim 1, wherein an opening of the well in the surface of each receptacle faces in substantially the same direction when the apparatus is in an open position and the openings oppose each other when the apparatus is in a closed position.

3. The apparatus of claim 1, wherein the strap is flexible and forms a flexure such that the strap is bendable about a midpoint between the pair of receptacles.

4. The apparatus of claim 3, wherein the strap permits the pair of receptacles to be oriented such that the opening of one of the wells is opposable the opening of the other well.

5. The apparatus of claim 1, wherein the relative sizes and dimensions of the well and the foam tablet are such that an interference fit occurs when the foam tablet is inserted into the well.

6. The apparatus of claim 1, further comprising a replaceable foam tablet having a dimension such that a portion of the foam tablet extends above the top of the well.

7. The apparatus of claim 1, wherein the foam tablet is an open cell resin foam.

8. The apparatus of claim 1, wherein a portion of at least one of the strap or the receptacle is ridged or roughened to facilitate gripping with fingers of the hand.

9. A method of treating a pierced body part, the method comprising:
providing a treatment device, comprising:
a pair of receptacles sized and dimensioned to accept a foam tablet in a well thereof; and
a strap joining the pair of receptacles such that an opening in the well in the surface of each receptacle faces in substantially the same direction when in an open position;
providing a foam tablet;
inserting the foam tablet in the well of the treatment device;
charging the foam tablet with a treatment liquid;
applying the foam tablet to the body area to be treated by folding the treatment device about a hinge or flexure formed by the strap into a closed position such that a free face of the foam tablet in the well of one receptacle is opposed to a free face of the foam tablet in the well of another receptacle.

10. A kit for treatment of a body piercing, comprising:
a treatment device including:
a pair of receptacles sized and dimensioned to accept a foam tablet in a well thereof; and
a strap joining the pair of receptacles; and
at least one of:
a foam tablet;
a treatment liquid; and
a treatment liquid dispenser.

11. The kit of claim 10, wherein the treatment liquid dispenser is one of a pipette, a medicine dropper or a nozzle of a liquid container.

12. The kit of claim 10, wherein the treatment liquid dispenser is capable of dispensing a predetermined quantity of the treatment liquid using a defined volume or a graduated marking.

13. A device for dispensing a treatment liquid, comprising:
a foam tablet,
wherein the tablet is shaped as a circular or substantially circular cross-section and having a height, the cross-section is sized and dimensioned to form an interference fit with an aperture in a holder adapted to receive the tablet, and the height of the tablet is greater that a depth of the aperture.

14. The device of claim 13, wherein the cross section is one of approximately an oval, an ellipse, a rectangle, or a square.

15. The device of claim 13, wherein the foam tablet is charged with a predetermined amount of a cleaning or disinfecting liquid.

16. The device of claim 15, wherein the liquid is one of saline solution, mild liquid anti-microbial or germicidal soap, or benzalkonium chloride.
